(19) United States
(12) Patent Application Publication
(10) Pub. No.: US 2022/0257811 A1
(43) Pub. Date: Aug. 18, 2022

(54) DISINFECTION DEVICE

(71) Applicant: Mallard Holdings LLC, Grand Prairie, TX (US)

(72) Inventors: Brian L. Mallard, Grand Prairie, TX (US); Jorge Emilio Molina, San Diego, CA (US); Lawrence D. Holt, II, Mabank, TX (US); Dorothy Anne Holt, Mabank, TX (US); Robert Kay, Thousand Oaks, CA (US); Graham Ross, Oceanside, CA (US)

(73) Assignee: Mallard Holdings LLC, Grand Prairie, TX (US)

(21) Appl. No.: 17/341,520

(22) Filed: Jun. 8, 2021

Related U.S. Application Data
(60) Provisional application No. 63/149,414, filed on Feb. 15, 2021.

(51) Int. Cl. A61L 2/10; A61L 2/26; A63D 5/10

(52) U.S. Cl. CPC A61L 2/10; A61L 2/26; A61L 2202/11; A61L 2202/122; A63D 5/10

(57) ABSTRACT

An apparatus for disinfecting an object is disclosed herein. The apparatus includes an enclosure having an interior, an entrance, an exit, and a path through the interior from the entrance to the exit along which the object travels. The object moves along the path from the entrance to the exit without support from a moving conveyance. The apparatus also includes a light source that is positioned within the interior and configured to emit ultraviolet light such that every exterior surface of object is exposed to the ultraviolet light as the object traverses the interior.

### CROSS-REFERENCE TO RELATED APPLICATIONS

This application claims priority to U.S. provisional application 63/149,414 filed on Feb. 15, 2021 and currently pending, which is hereby incorporated herein in its entirety.

### STATEMENT REGARDING FEDERALLY SPONSORED RESEARCH OR DEVELOPMENT

Not applicable.

### BACKGROUND

### Field

The present invention generally relates to the use of ultraviolet (UV) light to disinfect objects while in motion.

### Description of the Related Art

UV light is known to disrupt biological structures including bacteria and viruses. The germicidal effect of UVC light at sufficient intensity is known, as are the hazards of skin and eye damage after very short durations of exposure.

### SUMMARY

In certain embodiments, it is desirable to provide a system that disinfects items while moving along a defined path to improve the safety of users of the conveyance or items.

An apparatus for disinfecting an object is disclosed herein. The object has an exterior surface that can be touched by a user and an interior surface that is not accessible to be touched by the user. The apparatus includes an enclosure having an interior, an entrance, an exit, and a path through the interior from the entrance to the exit along which the object travels. The object moves along the path from the entrance to the exit without support from a moving conveyance. The apparatus also includes a light source that is positioned within the interior and configured to emit ultraviolet light such that the exterior surface of object is exposed to the ultraviolet light as the object traverses the interior.

An apparatus for disinfecting an entire external surface of a bowling ball while it is rolling along a ball return track of a bowling alley is disclosed herein. The apparatus includes an enclosure configured to be disposed proximate to the track such that the track traverses an interior of the enclosure. The apparatus also includes a light source coupled to the enclosure and configured to emit ultraviolet light. The total energy of the ultraviolet light received at the entire external spherical surface of the bowling ball, as the bowling ball passes through the enclosure, is sufficient to inactivate at least a threshold percentage of a target infectious agent.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates an exemplary escalator.

FIG. 2A depicts an exemplary location of a disinfecting apparatus, according to certain aspects of the present disclosure.

FIG. 2B depicts the contact and non-contact surfaces of an exemplary escalator handrail, according to certain aspects of the present disclosure.

FIGS. 3-5 depict exemplary embodiments of a disinfection apparatus, according to certain aspects of the present disclosure.

FIG. 6 depicts a block diagram of an exemplary disinfection system, according to certain aspects of the present disclosure.

FIGS. 7-9 depict exemplary arrangements of internal elements of disinfection apparatus, according to certain aspects of the present disclosure.

FIG. 10 depicts exemplary placements of disinfection apparatus, according to certain aspects of the present disclosure.

FIG. 11A is an exemplary plot of a typical spatial distribution of light emitted by an LED, according to certain aspects of the present disclosure.

FIG. 11B is a chart showing bacterial cell kill rate by 222 and 254 nm light, according to certain aspects of the present disclosure.

FIGS. 12A and 12B depict components of letter sorting systems, according to certain aspects of the present disclosure.

FIG. 13 depicts an exemplary disinfection device configured to illuminate an outside surface of an escalator handrail, according to certain aspects of the present disclosure.

FIG. 14 depicts an exemplary disinfection device configured to illuminate an external surface of a bowling ball, according to certain aspects of the present disclosure.

FIG. 15 depicts an exemplary disinfection device comprising an unpowered support, according to certain aspects of the present disclosure.

FIG. 16 depicts an exemplary disinfection device comprising a tumbling actuator, according to certain aspects of the present disclosure.

FIGS. 17A-17C depict exemplary disinfection devices for use with a fuel pump, according to certain aspects of the present disclosure.

FIG. 18 depicts exemplary disinfection devices for use within an operating room, according to certain aspects of the present disclosure.

### DETAILED DESCRIPTION

The following description discloses embodiments of a disinfection apparatus.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form to avoid obscuring the concepts of the subject technology. Like, or substantially similar, components are labeled with identical element numbers for ease of understanding.

As used within this disclosure, the term “frequency” means a rate of oscillation that has a nominal value and may have an associated band of variation around the nominal value. For example, light described as having a frequency may include light having a range of frequencies that includes the stated value.

As used within this disclosure, the term “light” refers to electromagnetic radiation having wavelengths in the range of 10 nm-1 mm.

As used within this disclosure, the term “monochromatic” means that there is a nominal wavelength for the light with the majority of the energy being within a narrow band around this nominal wavelength.

As used within this disclosure, the phrase “ultraviolet light” refers to electromagnetic radiation having wavelengths in the range of 10-400 nm, with subtypes of UV-C (100 nm to 280 nm), UV-B (280 nm to 315 nm), and UV-A (315 nm to 400 nm). UV radiation is present in sunlight and is also produced by electric arcs and specialized lights such as mercury-vapor lamps and black lights.

As used within this disclosure, the phrase “infectious agent” refers to any organism that can produce disease including bacteria, germs, viruses, and other pathogens or pathogen hosts.

As used within this disclosure, the term “conveyor” refers to a powered moving endless support belt having a direction of motion of the belt defining a path and/or a series of rotating powered rollers arranged along a similar path that supports material or objects on an upper surface of the belt or rollers. Activation of the conveyor moves the material or objects along the path.

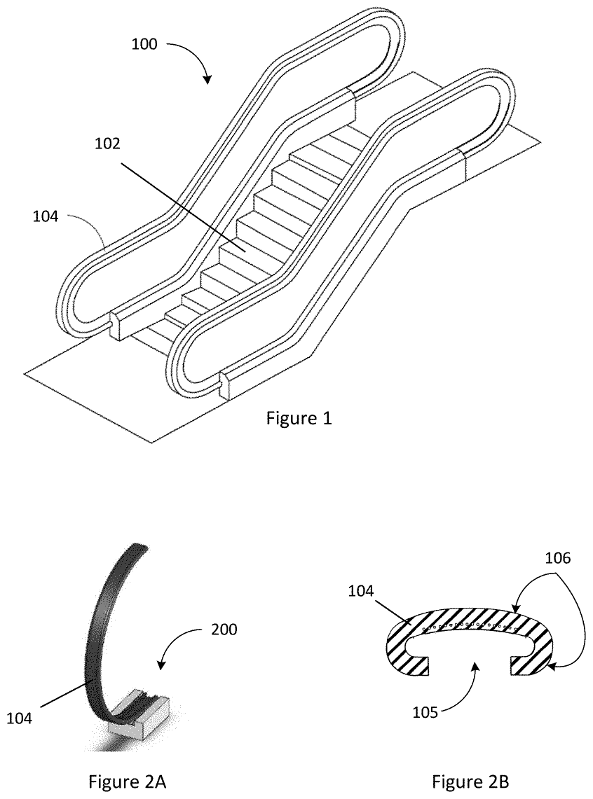

FIG. 1 illustrates an exemplary escalator 100 with treads 102 and a handrail 104. The handrail 104 is conventionally provided as an endless band.

FIG. 2A depicts an exemplary location of a disinfecting apparatus 200, according to certain aspects of the present disclosure. In this example, the disinfection apparatus is located proximate the point where the handrail 104 emerges from the understructure and is first available to be touched by a user.

FIG. 2B depicts the contact and non-contact surfaces of an exemplary escalator handrail 104, according to certain aspects of the present disclosure. Surfaces 106 are exterior surfaces that can be touched by a user and surface 105 is an interior surface that is not accessible to be touched by the user in normal usage.

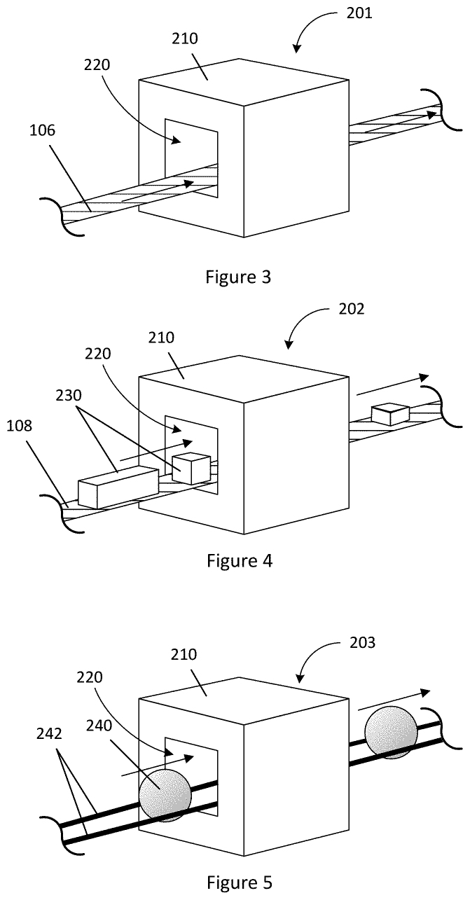

FIG. 3 depicts an exemplary embodiment of a disinfection apparatus 201, according to certain aspects of the present disclosure. This embodiment comprises an enclose 210 that accepts a continuous conveyance 106, for example an escalator handrail, into an entrance 220 and the conveyance emerges from an exit (not visible in FIG. 4). Within the apparatus 201, a portion of the conveyance 106 is exposed to germicidal UV light to disinfect the exposed surface. The length of the conveyance that is exposed to UV light within the apparatus 201 is selected based on the speed of the conveyance 106 and the intensity of the UV light at the exposed surface of the conveyance 106 so as to ensure that a representative point on the exposed surface of the conveyance 106 is exposed long enough at the threshold intensity to achieve a threshold “kill rate” for one or more infectious agents.

FIG. 4 depict an exemplary embodiment of a disinfection apparatus 202, according to certain aspects of the present disclosure. This embodiment comprises an enclose 210 that accepts a continuous conveyance 108, for example a conveyor belt, on which a plurality of objects 230, for example shipping boxes or pieces of mail, into an entrance 220 and the conveyance 108 and items 230 emerges from an exit (not visible in FIG. 4).

FIG. 5 depicts an exemplary embodiment of a disinfection apparatus 203, according to certain aspects of the present disclosure. This embodiment comprises an enclosure 210 that accepts series of objects 240, for example bowling balls, moving along a defined path established by rails 242 into an entrance 220 and the objects 240 emerge from an exit (not visible in FIG. 5) on the rails 242.

The enclosure 210 has an interior and, in this embodiment, the rails 242 define a path through the interior from the entrance 220 to the exit along which the object 240 travels. In this embodiment, the rails 242 are fixed and the object moves along the path from the entrance to the exit without support from a moving mechanism, for example the conveyance 106 of FIG. 3. In certain embodiments, the object 240 enters the enclosure 210 with a velocity sufficient to carry the object to the exit of the enclosure without application of force or moving support within the enclosure 210.

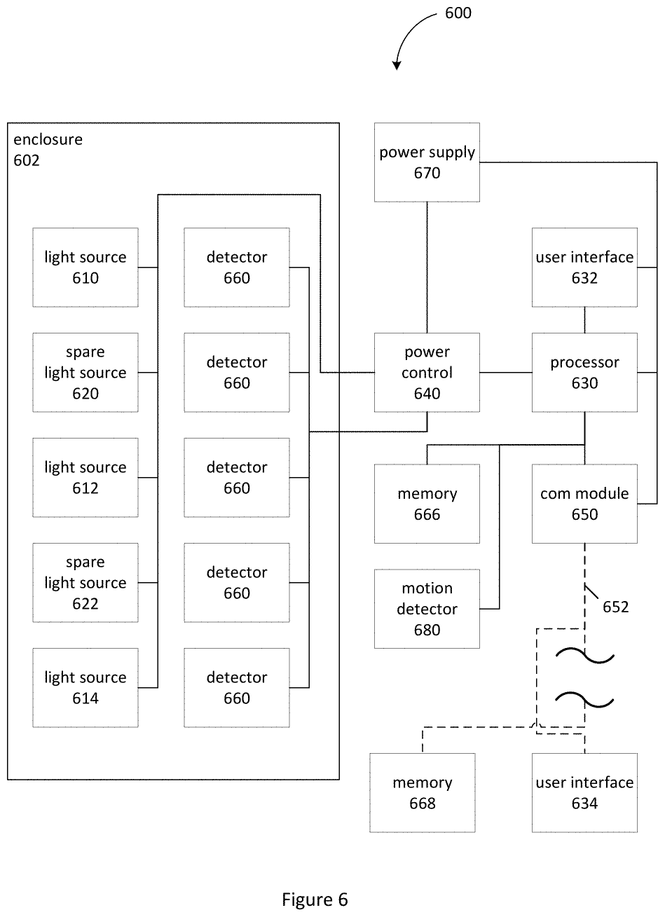

FIG. 6 depicts a block diagram of an exemplary disinfection system 600, according to certain aspects of the present disclosure. An enclosure 602 contains a first plurality of light sources 610, 620 and a second plurality of light detectors 660. The light sources 610, 620 are connected to a power control 640 that is configured to activate, deactivate, or otherwise interact individually with each of the items 610, 620, 660. In certain embodiments, the power module 640 is configured to detect whether a light source 610, 620 is functioning, for example drawing current above a threshold. In certain embodiments, the detectors 660 are configured to detect whether a particular light source 610, 620 is emitting light and/or the strength of the emitted light.

In certain embodiments, the selection of which light sources 610, 620 are activated is based in part on the goal of continuously disinfecting the conveyance. A failure of a light source 610 would normally reduce the total energy of light delivered to the surface of the conveyance. In certain embodiments, detection the failure by one or more of the power control 640 and a detector 660 will enable the processor 630 to select a spare light source 620 and command the power control 640 to activate the selected light source 620.

In certain embodiments, a spare light source 620 that is activated be considered to convert to a light source 610 and remain in service. Replacement or repair of the failed light source 610 may be considered to convert the replaced//repaired light source to a spare light source 620. In certain embodiments, the processor 630 is configured to retain information about the current state of light sources, for example which units are currently a “in use” light source 610 and which units are a “spare” light source 620 and record this information in one or both of memories 666, 668. The processor may further be configured to record the “on time” of each light source 610 and provide this information to a user interface 632, 634.

In certain embodiments, the light sources 610, 620 emit ultraviolet light. In certain embodiments, the emitted light comprises light with wavelengths within the 200-580 nm band. In certain embodiments, the emitted light comprises light with wavelengths in the band of one of the subtypes of UV-C, UV-B, and UV-A. In certain embodiments, the light sources 610, 620 are shortwave fluorescent tubes using fused quartz tubes without a phosphor coating that emit light primarily at approximately 254 nm. In certain embodiments, the light sources 610, 620 emit light primarily in a band of 260-280 nm. In certain embodiments, the light sources 610, 620 emit light primarily in a band of 260-265 nm. In certain embodiments, the light sources 610, 620 emit light primarily in a band of 220-225 nm, referred to herein as “far-UVC” light. In certain embodiments, the light sources 610, 620 are one of light emitting diodes (LEDs), mercury lamps, or gas discharge bulbs. In certain embodiments, the light sources 610, 620 are excimer lamps that emit light with a peak intensity at approximately 222 nm.

Far-UVC light has a particular advantage, compared to other frequency bands of UV light, in that this frequency band of light penetrates only a very short range in biological material. Conventional germicidal UV light is typically around 254 nm, within the UVC band, which is effective but can cause severe burns of the skin and eye injuries (photokeratitis) after an exposure of seconds to minutes. Far-UVC light cannot penetrate the dead-cell layer at the surface of human skin nor can it penetrate into human eyes, making this frequency intrinsically safe at higher energy levels that are acceptable other types of UV light.

The total energy delivered to a surface determines the efficacy of disinfection. Far-UVC light has been shown to inactivate 99.9% of aerosolized coronavirus 229E and OC43 at doses of 1700 and 1200 μJ/cm², respectively. Inactivation of other infectious agents can be determined by conventional experiments varying the intensity and exposure time and measuring the decrease in active organisms. Dosage is calculated using the formula:

dose(μJ/cm²)=(μW·sec/cm²)=intensity(μW/cm²)×exposure time(sec)

It is generally considered impossible to achieve a 100% kill rate for one or more target infectious agents on a surface at practical exposure intensities and times. Disinfection is defined as a reduction of at least a threshold percentage of an active infectious agent. Disinfection is generally assumed to cause the number of living microorganisms to decay exponentially. Disinfection systems may be characterized by a decimal reduction dose, or “D-value,” that is the dose required to achieve a “log reduction,” i.e. to kill 90% (or 1 log) of a target infectious agent. A reduction of 99.99% is commonly referred to as a “four 9 s” reduction. In a system wherein an object is repeatedly exposed to a disinfecting apparatus while in use, even a reduction of a conventionally low percentage, for example a 50% reduction, may be sufficient to maintain the safety of the users.

In certain embodiments, the system 600 includes one or more of a power supply 670, a processor 630 coupled to user interfaces 632, 634 and memories 666, 668 and a communication (“com”) module 650. In certain embodiments, the com module 650 is coupled to the user interface 634 and/or the memory 668 via a telecommunication system 652, for example a network comprising transmission lines and routers and processors configured to transmit information using a protocol such as ethernet. One of ordinary skill in the art will be familiar with alternate methods of remote communication and such alternate embodiments are not presented herein.

In certain embodiments, the light sources 610 are activated and the light sources 620 are deactivated. In certain embodiments, detection of failure of a light source 610 by the power control 640, for example by detecting a change in current drawn by the light source 610 or a change in the intensity of the light emitted by the light source 610 as detected by a detector 660, will cause the power control to switch on a space light source 620. In certain embodiments, the power control 640 provides a signal to the processor 630 of the failure and the processor 630 sends a signal through the com module 650 and telecommunication system 652 to a remote user interface 634. In certain embodiments, this user interface 634 is accessible by a service company who may schedule a service call to replace the failed light source 610.

In certain embodiments, a motion detector 980 is provided to detect whether the conveyance being disinfected is in motion and connected to the processor 630. If the motion detector determines that the conveyance stops moving, the process 630 may command the power control 640 to deactivate one or more of the light sources 610, 620. If the motion detector 680 determines that the conveyance changes from a stopped state to a moving state, the processor 630 may command the power control 640 to activate one or more of the light sources 610, 620. In certain embodiments, the deactivation and/or the activation process may comprise a time delay, a modulation of the power provided to the light sources 610, 620, or a gradual change between states of “on” or “off” of the light sources 610, 620.

In certain embodiments, instructions are stored on at least one of the memories 666, 668 that, when loaded on and executed by the processor 630, cause the processor to determine one or more of the state of a component, the time-in-service for a component, or other attribute of the system 600.

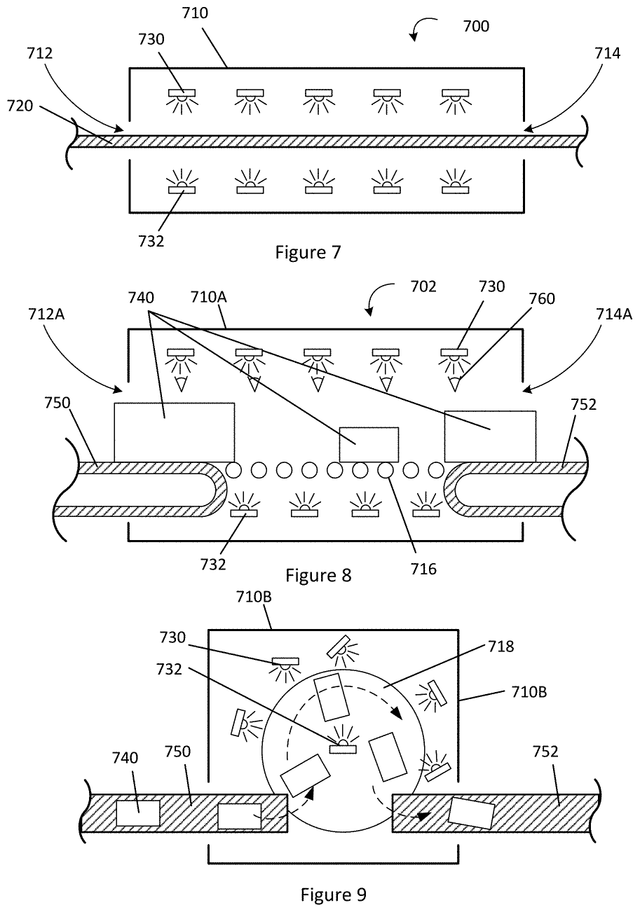

FIG. 7 depicts a side view of an exemplary arrangement of internal elements of disinfection apparatus 700, according to certain aspects of the present disclosure. In this embodiment, the continuous conveyance 720 passes between a first set of light sources 730 that disinfect the top side of conveyance 720 and a second set of light sources 732 that disinfect the underside of the conveyance 720.

FIG. 8 depicts an exemplary arrangement of internal elements of disinfection apparatus 702, according to certain aspects of the present disclosure. In this embodiment, the objects 740 are provided on a input conveyance 750 and transferred to an internal system 716, for example a plurality of powered rollers, that moves the objects 740 between light sources 730 and light sources 732 to an output convenance 752. The light sources 732 shine between the elements of the transfer system 716 to disinfect the underside of the objects. Detectors 760 are shown in exemplary positions to monitor and measure the intensity of the light emitted by the light sources 730. In other embodiments, additional detectors 760 monitor light sources 732. A single detector 760 may monitor one or more light sources 730, 732.

FIG. 9 depicts an exemplary arrangement of internal elements of disinfection apparatus 704, according to certain aspects of the present disclosure. In this embodiment, the objects 740 are provided on a input conveyance 750 and transferred to an internal system 718, for example a powered turntable, that moves the objects 740 between light sources 730 and light source 732 to an output convenance 752. In certain embodiments, the light sources 730, 732 are individually positioned and oriented so as to preferentially disinfect a selected portion of an object, for example the front, outside (with respect to the center of the turntable 718), the inside, or the back. Additional light sources (not shown in FIG. 9, may be positioned above or below the internal system 718 to disinfect the top and/or bottom of the objects 740.

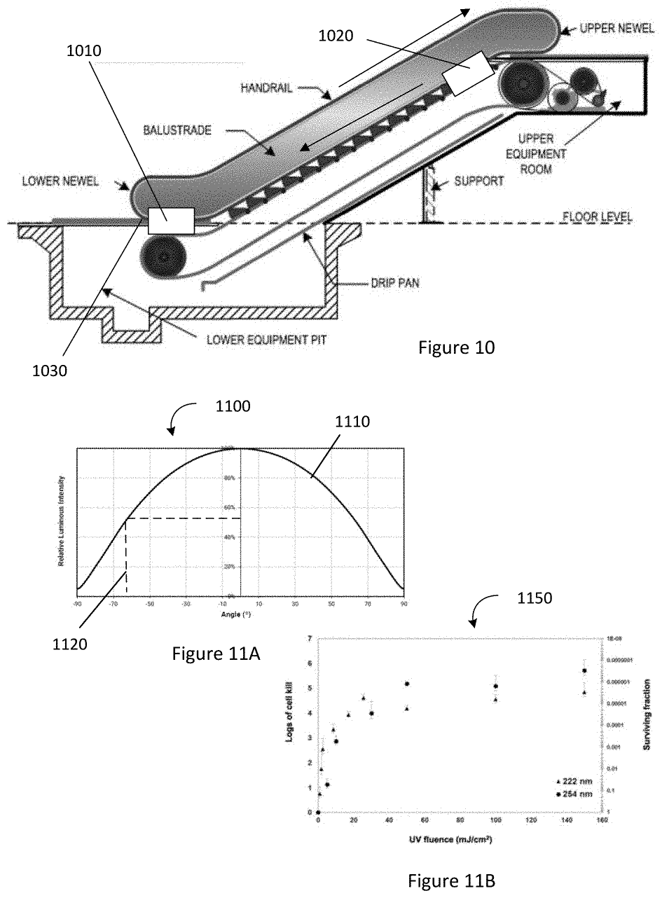

FIG. 10 depicts exemplary placements 1010, 1020 of disinfection apparatus in an escalator system 1000, according to certain aspects of the present disclosure. Location 1010 will present a freshly disinfected surface of the handrail at the point of emergence 1030 of the handrail. Location 1020 offers the ability to disinfect the handrail immediately after last contact by a user, thereby maintaining the length of the handrail from location 1020 to 1030 in a “clean” condition.

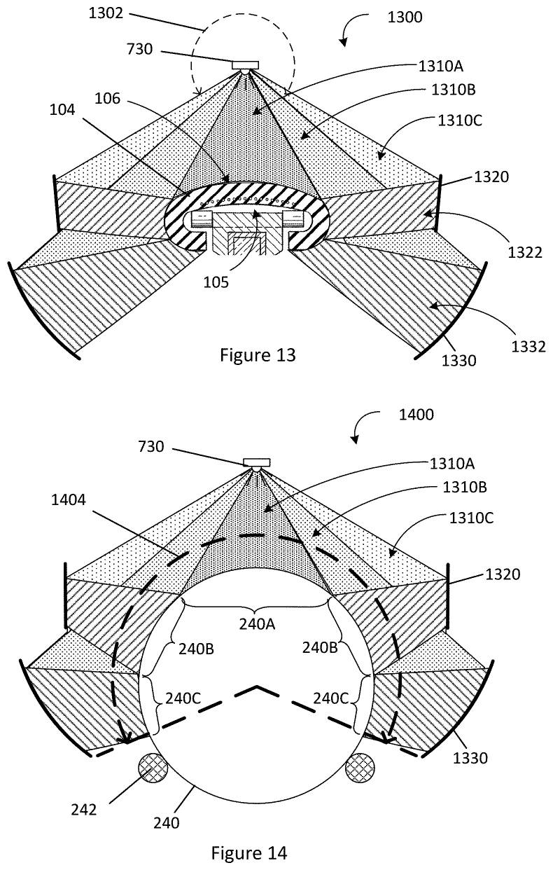

FIG. 11A is an exemplary plot 1100 of a typical spatial distribution of light emitted by an LED, according to certain aspects of the present disclosure. In this example, the intensity of the emitted light at an angle of ˜60° from the centerline of emission is approximately 50% of the intensity on the centerline. This can be used to define the angle of an “effective beam” of light emitted by an LED, wherein the value at the edge is considered to be the minimum energy within the beam. While light is emitted at angles greater than the selected angle, the “beam of light” emitted by a light source can be considered to be limited to the +/−selected angle. This is discussed further with respect to FIGS. 13 and 14.

FIG. 11B is a chart showing the bacterial cell killing by 222 and 254 nm light, according to certain aspects of the present disclosure. This chart is copied from FIG. 2 of the paper “Manuela Buonanno et al., Germicidal Efficacy and Mammalian Skin Safety of 222-nm UV Light, Radiation Research 2017 April; 187(4): 483-491.” The chart plots the cell killing of methicillin-resistance Staphylococcus aureus (MRSA), plotted as the −log 10[surviving fraction], for different levels of total delivered energy. The authors show that light in the range of 222 nm kills bacteria efficiently regardless of their drug-resistant proficiency, but without the skin-damaging effects associated with conventional germicidal UV light of 254 nm.

Conventional wisdom is that the optimum wavelength for the destruction of biological matter occurs close to 260 nm. Dosages for a 90% kill of most bacteria and viruses is in the range of 20-80 J/m². In certain applications where tougher organisms may be present, for example disinfection of potable water, a total dose of 250 J/m² is advised. One drawback of the conventional approach to using UVC light to disinfect surfaces is that UVC light is hazardous to most living things. Skin exposure can produce rapid sunburn and skin cancer. Exposure of the eyes to UVC light can produce extremely painful inflammation of the cornea and temporary or permanent vision impairment, up to and including blindness. The use of UV light of approximately 222 nm, and excluding light above 225 nm, is preferable and effective.

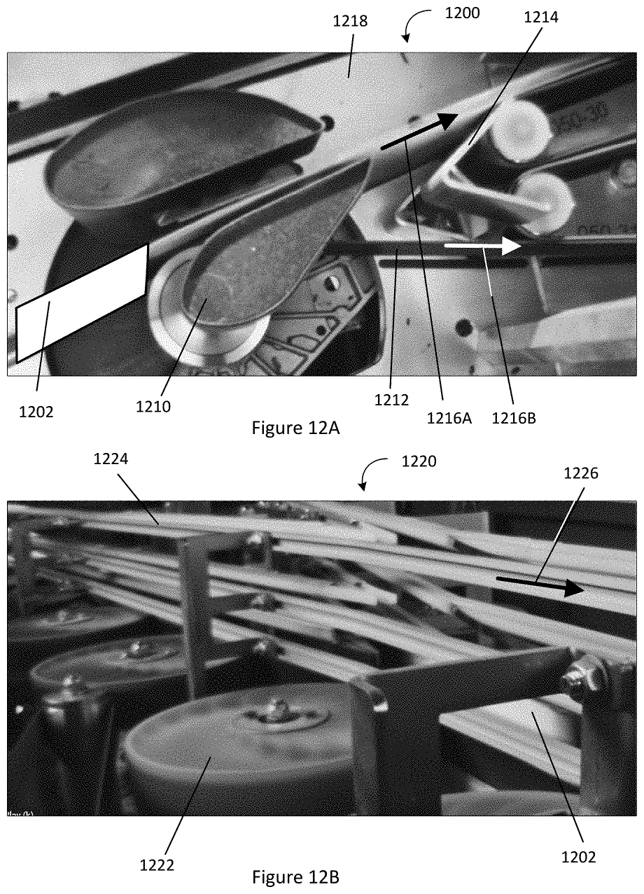

FIG. 12A depicts components of a letter sorting system 1200, according to certain aspects of the present disclosure. The entire outside of a letter 1202, or similar envelopes as well as packages, is consider an exterior surface that can be touched by a person while handling the envelope 1202. A letter 1202 is shown in a position along the path of travel of letters moving through the system 1200. Letters slide over a fixed support surface 1218 as they pass through the system 1200. Flippers 1210 direct a letter onto one of the two output directions indicated by arrows 1216A and 1216B. The fixed side guide 1214 defines a portion of each path 1216A, 1216B. The position of flippers 1210 in FIG. 12A will direct a letter onto path 1216A. An actuator, in the form of a side belt 1212, is shown proximate to the path 1216B and disposed such that the side belt 1212 will contact the side of a letter on path 1216B. The side belt 1212 moves laterally when actuated so as to apply a lateral force to the side of the letter and thereby urge the letter along the path 1216B.

FIG. 12B depict components of a letter handling system 1220, according to certain aspects of the present disclosure. This embodiment of a system 1200 includes multiple fixed side guides 1224 and multiple actuators in the form of side rollers 1222 that are disposed to contact the side of a letter 1202 passing through system 1220. The side roller 1222 rotates laterally when actuated so as to apply a lateral force to the side of the letter 1202 and thereby urge the letter 1202 along the path 1226.

FIG. 13 depicts an exemplary disinfection device 1300 configured to illuminate an outside surface 106 of an escalator handrail 104, according to certain aspects of the present disclosure. The beam of light emitted by light source 730 is shown as subregion beams 1310A, 1310B, 1310C that are collectively considered the effective beam as discussed with respect to FIG. 11. The angular limit of the effective beam is indicated by angle 1302, which is the complement of the included angle of the beam 1310A, 1310B, 1310C, wherein beams 1310B and 1310C are present on both sides of beam 1310A. Beam 1310A impinges directly on a portion of the exterior surface 106. Beam 1310B is reflected by reflector 1330 to a secondary beam 1332 toward a shadowed portion of surface 106 that does not have direct line-of-sight from light source 730. In certain embodiments, reflector 1330 comprises a concave surface such that secondary beam 1332 concentrates the light to increase the intensity of the light at the surface 106. Beam 1310C is reflected by reflector 1320 to a secondary beam 1322 toward a second shadowed portion of surface 106. In certain embodiments, reflector 1320 comprises a flat surface. In certain embodiments, one or more of reflector 1320 and reflector 1330 comprises a convex surface that disperses incident light. The system 1300 is configured such that every point on a surface 106 of handrail 104 is exposed to the ultraviolet light as the object traverses the system 1300. In certain embodiments, the interior 105, which does not come into contact with users of the handrail 104, is not exposed to light from light source 730.

FIG. 14 depicts an exemplary disinfection device 1400 configured to illuminate an external surface of a bowling ball 240, according to certain aspects of the present disclosure. In this example, the bowling ball 240 rolls along a pair of fixed rails 242. As it passes under light source 730, beam 1310A directly illuminates a strip 240A of the surface of the bowling ball 240 that includes the entire circumference around a horizontal axis through the center of the bowling ball 240. Beams 1310B are reflected by reflectors 1320 to illuminate strips 240B on each side of the bowling ball 240, again covering a continuous strip around the bowling ball 240. Beams 1310C are similarly reflected by reflectors 1330 to illuminate strips 240C on each side of the bowling ball 240, again covering a continuous strip around the bowling ball 240. Collectively, beams 1310A, 1310B, and 1310C cover an angle 1404 around a plane through the center of the bowling ball 240. If this angle 1404 is greater than 180°, then rotation of the bowling ball 240 as it moves along the path defined by rails 242 within the device 1400 will expose the entire surface of the bowling ball 240 to light from the light source 730. In certain embodiments, small features, for example the finger holes of a bowling ball, are not considered to be part of the external surface being disinfected. In certain embodiments, the small features are important to disinfect and the system is configured to disinfect them at the same level as the external surface being disinfected.

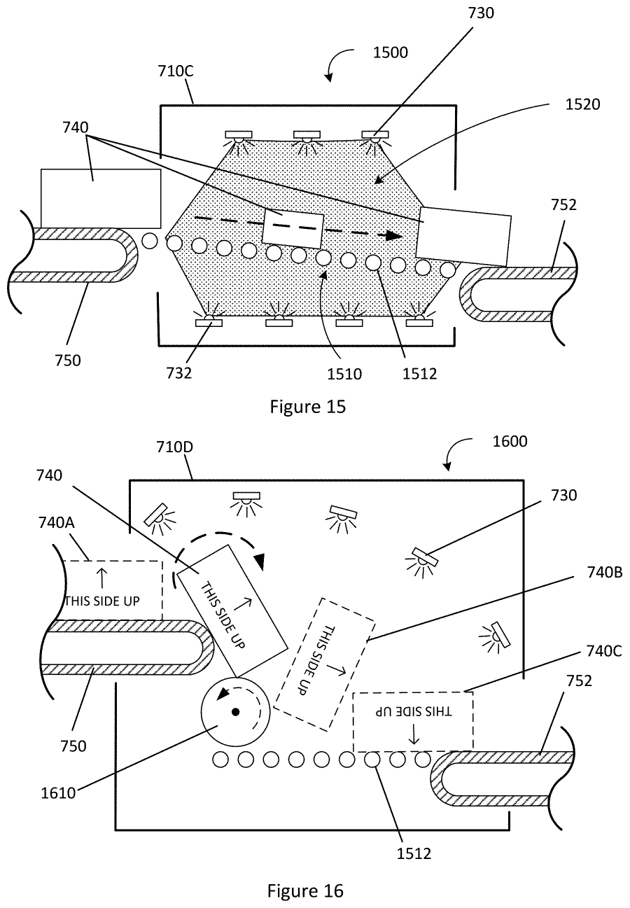

FIG. 15 depicts an exemplary disinfection device 1500 comprising an unpowered support 1510, according to certain aspects of the present disclosure. In the embodiment shown in FIG. 15, the support 1510 comprises multiple unpowered rolling elements 1512 arranged to form an inclined path from an entrance conveyance 750 to an exit conveyance 752. An object 740 that is transferred from the entrance conveyance 750 onto support 1510 will move under the influence of gravity, i.e. without support of a moving conveyance, along the path to the exit conveyance 752. In this example, the rolling elements 1512 are not considered to form a “moving conveyance” as each provides support at a fixed location along the path of the object 740. In certain embodiments, an entrance to enclosure 710C is elevated above an exit of enclosure 710C with respect to gravity such that the object 740 is urged along the path by gravity.

In certain embodiments, entrance conveyance 750 may impart a velocity to object 740 and the object 740 will traverse the path from the entrance to the exit when the object 740 enters the entrance at a velocity at or above a defined minimum velocity.

Light sources 730, 732 are disposed above and below the path of support 1510 to create an illuminated region 1520 wherein objects 740 passing through device 1500 are exposed to light from the light sources 730, 732. The portion of support 1510 that is within the illuminated region 1520 defines an illuminated length of the path of objects 740. In certain embodiments, the device 1500 comprises interior baffles (not shown in FIG. 15) that additionally define the illuminated region and the illuminated length. In certain embodiments, an object 740 moves along the illuminated length of the path without support from a conveyance. As the supports 1512 do not move with the object 740, the entire underside of object 740 is exposed to light from one or more of the light sources 732 as the object 740 passes over a gap between supports 1512.

FIG. 16 depicts an exemplary disinfection device 1600 comprising a tumbling actuator 1610, according to certain aspects of the present disclosure. The tumbling actuator 1610 is positioned such that, in conjunction with the motion provided by other elements such as the entrance conveyance 750, an object 740 is repositioned to provide exposure of all surfaces of the object 740 to light from the light sources 730. In this exemplary sequence, an object 740 is first at dashed-line position 740A on entrance conveyance 750. At the solid-line position of object 740, the object 740 has tumbled off the end of entrance conveyance 750 and come into contact with the surface of tumbling actuator 1610 that, in this example, is counter-rotating as indicated by the arrow. This applies a force to object 740 that further rotates object 740 as indicated by position 740B until it reaches an inverted position indicated by position 740C. In certain embodiments, the direction of rotation my be perpendicular to the path through the device 1600. In certain embodiments, the object 740 may not be fully inverted but simply repositioned sufficient to exposure surfaces previous shadowed from the light sources 730. In certain embodiments, the support elements 1512 together form a horizontal path and a side force is applied by actuators (not shown in FIG. 16) that contact a side of the objects 740. In certain embodiments, the support elements 1512 together form a path that is inclined toward the exit conveyance 752.

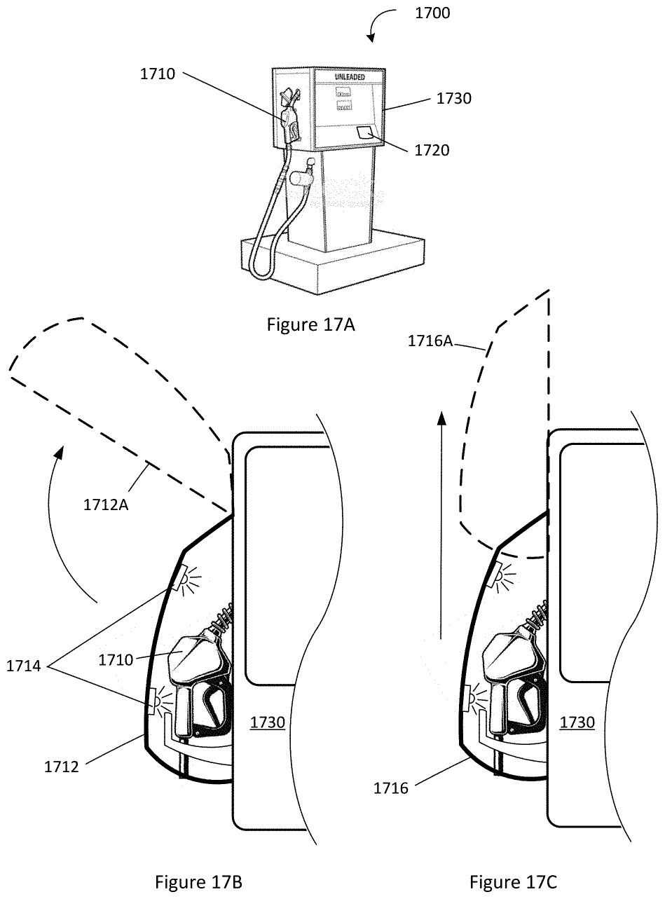

FIG. 17A depicts a fuel pump 1700 with a body 1730, a dispensing handle 1710 and a user interface 1720, e.g. a keypad, according to certain aspects of the present disclosure.

FIG. 17B depicts an exemplary embodiment of a cover 1712 disposed over the dispensing handle 1710 when the handle 1710 is stored on the body 1730. In this embodiment, the cover 1712 is hinded to the body 1730 with a closed position indicated by the position of cover 1710 and an open position 1712A shown in dashed line. One or more light sources 1714 are disposed on an interior of the cover such that a portion of the surfaces of the handle 1710 are exposed to disinfecting light emitted by the sources 1714. In operation, the light sources 1714 are activated when the cover 1712 is returned from an open position 1712A to the closed position shown as 1712. In certain embodiments, the light sources 1714 are activated for a predetermined amount of time.

FIG. 17C shows a second exemplary embodiment wherein the cover 1716 slides upward to an open position 1716A, shown in dashed line.

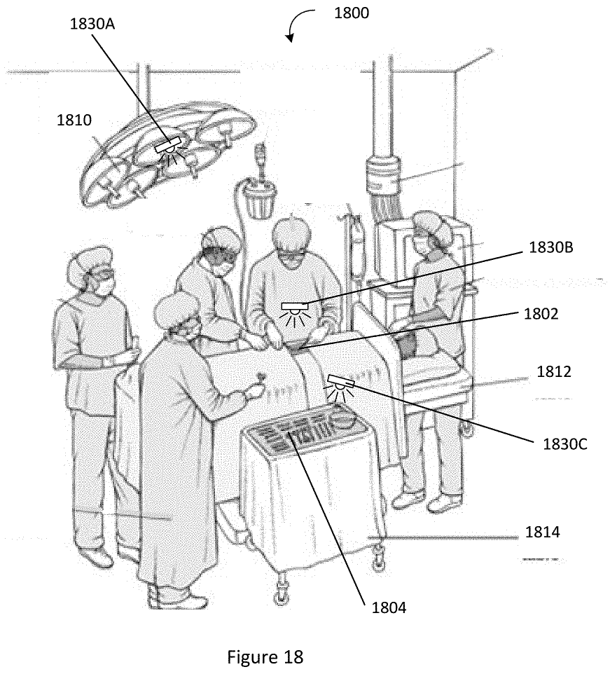

FIG. 18 depicts exemplary disinfection devices for use within an operating room 1800, according to certain aspects of the present disclosure. It is desirable to maintain a sterile environment throughout the room 1800 and particularly with regard to an open surgical site 1802 and instruments 1804 to be used by the surgeon. The operating room 1800 is meticulously cleaned prior to each surgery and all instruments and other supplies, e.g. surgical drapes, sponges, gloves, are sterilized in advance. Before the surgery starts, the sterile goods are placed on the tables or stands where they will be used to avoid having to move them from one place to another during the surgery. The linen or drape pack is typically on the instrument table, aka the “backtable.” The gown pack is placed on the prep table. The Mayo tray is placed on the Mayo stand. The sterile basin set is set into the ring stand. The prep set placed on the prep table. The instrument set is placed on a ring stand or table.

In an embodiment, a light source 1830A that emits disinfecting light is disposed adjacent to existing room lights 1810. In certain embodiments, the light source 1830A emits eye-safe UV light, for example around 222 nm, so that the staff are not adversely affected. In certain embodiments, light source 1830A comprises focusing optics configured to direct the disinfecting light to a particular site, e.g. the surgical site 1802.

In an embodiment, a light source 1830B that emits disinfecting light is disposed proximate to the surgical site 1802 so as to selectively illuminate the site 1802. Support structure for light source 1830B is not shown in FIG. 18 to improve the clarity of the illustration. In certain embodiments, the light source 1830B may be provided as a headlamp or as part of glasses (not shown in FIG. 18) worn by the surgeon.

In an embodiment, a light source 1830C that emits disinfecting light is disposed proximate to the instruments and supplies 1804 provided on the back table 1814. It will be apparent that this arrangement may be duplicated for other auxiliary equipment, carts, and furniture within the operating room to disinfect addition stock of instruments and supplies as well as equipment touched by the surgeon or staff. In certain embodiments, light sources 1830 (encompassing embodiments 1830A, 1830B, 1830C and variations thereof) are disposed inside storage cases and cabinets (not shown in FIG. 18) to maintain the sterility of items stored within.

### Aspects

Aspect 1: An apparatus for disinfecting an object having an exterior surface that can be touched by a user and an interior surface that is not accessible to be touched by the user, comprising: an enclosure comprising an interior, an entrance, an exit, and a path through the interior from the entrance to the exit along which the object travels, wherein the object moves along the path from the entrance to the exit without support from a moving conveyance; and a light source disposed within the interior and configured to emit ultraviolet (UV) light such that the exterior surface of object is exposed to the UV light as the object traverses the interior while the interior surface is not exposed to the UV light.

Aspect 2: The apparatus of aspect 1, wherein the path comprises an illuminated region arranged such that a total energy of the ultraviolet light received at every exterior surface of the object while within the illuminated region is sufficient to inactivate at least a threshold percentage of a target infectious agent.

Aspect 3: The apparatus of aspect 2, further comprising a fixed support surface over which the object moves while within the illuminated region.

Aspect 4: The apparatus of aspect 3, further comprising a fixed side guide that defines a portion of the path.

Aspect 5: The apparatus of aspect 3, further comprising an actuator disposed proximate to a side of the path and configured to urge the object along the path.

Aspect 6: The apparatus of aspect 5, wherein the actuator is configured to apply a lateral force to a side of the object.

Aspect 7: The apparatus of aspect 6, wherein the actuator is one or more of a side belt and a side roller, wherein: the side belt or the side roller are configured to contact the side of the object; the side belt moves laterally when actuated; and the side wheel rotates laterally when actuated.

Aspect 8: The apparatus of aspect 2, further comprising a non-powered support over which the object moves while within the illuminated region.

Aspect 9: The apparatus of aspect 8, wherein the entrance is elevated above the exit such that gravity is the sole force urging the object along the path from the entrance to the exit.

Aspect 10: The apparatus of aspect 8, wherein the path is configured such the object will traverse the illuminated region when the object enters the illuminated region at or above a defined minimum velocity.

Aspect 11: The apparatus of aspect 8, wherein the fixed support comprises a non-powered rolling element over which the object rolls.

Aspect 12: The apparatus of aspect 8, further comprising a tumbling actuator disposed proximate to the path and configured to reorient the object.

Aspect 13: The apparatus of aspect 2, further comprising a reflector disposed within the interior and configured to redirect a portion of the ultraviolet light onto a shadowed surface of the object.

Aspect 14: The apparatus of aspect 1, wherein the ultraviolet light is limited to wavelengths within the band of 220-225 nm such that the apparatus does not pose a hazard to people proximate to the apparatus while in operation.

Aspect 15: An apparatus for disinfecting an entire external surface of a bowling ball while it is rolling along a ball return track of a bowling alley, comprising: an enclosure configured to be disposed proximate to the track such that the track traverses an interior of the enclosure; and a light source coupled to the enclosure and configured to emit ultraviolet light; wherein a total energy of the ultraviolet light received at the entire external spherical surface of the bowling ball, as the bowling ball passes through the enclosure, is sufficient to inactivate at least a threshold percentage of a target infectious agent.

Aspect 16: The apparatus of aspect 15, further comprising a reflector coupled to the enclosure and configured to reflect a portion of the ultraviolet light toward the track so as to illuminate a portion of the bowling ball that is shadowed from the ultraviolet light coming directly from the light source.

Headings and subheadings, if any, are used for convenience only and do not limit the invention.

Reference to an element in the singular is not intended to mean “one and only one” unless specifically so stated, but rather “one or more.” Use of the articles “a” and “an” is to be interpreted as equivalent to the phrase “at least one.” Unless specifically stated otherwise, the terms “a set” and “some” refer to one or more.

Terms such as “top,” “bottom,” “upper,” “lower,” “left,” “right,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Although the relationships among various components are described herein and/or are illustrated as being orthogonal or perpendicular, those components can be arranged in other configurations in some embodiments. For example, the angles formed between the referenced components can be greater or less than 90 degrees in some embodiments.

Although various components are illustrated as being flat and/or straight, those components can have other configurations, such as curved or tapered for example, in some embodiments.

Pronouns in the masculine (e.g., his) include the feminine and neuter gender (e.g., her and its) and vice versa. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “operation for.”

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such as an embodiment may refer to one or more embodiments and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

Although embodiments of the present disclosure have been described and illustrated in detail, it is to be clearly understood that the same is by way of illustration and example only and is not to be taken by way of limitation, the scope of the present invention being limited only by the terms of the appended claims.

What is claimed is:

1. An apparatus for disinfecting an object having an exterior surface that can be touched by a user and an interior surface that is not accessible to be touched by the user, comprising:
an enclosure comprising an interior, an entrance, an exit, and a path through the interior from the entrance to the exit along which the object travels, wherein the object moves along the path from the entrance to the exit without support from a moving conveyance; and
a light source disposed within the interior and configured to emit ultraviolet (UV) light such that the exterior surface of object is exposed to the UV light as the object traverses the interior while the interior surface is not exposed to the UV light.

2. The apparatus of claim 1, wherein the path comprises an illuminated region arranged such that a total energy of the ultraviolet light received at every exterior surface of the object while within the illuminated region is sufficient to inactivate at least a threshold percentage of a target infectious agent.

3. The apparatus of claim 2, further comprising a fixed support surface over which the object moves while within the illuminated region.

4. The apparatus of claim 3, further comprising a fixed side guide that defines a portion of the path.

5. The apparatus of claim 3, further comprising an actuator disposed proximate to a side of the path and configured to urge the object along the path.

6. The apparatus of claim 5, wherein the actuator is configured to apply a lateral force to a side of the object.

7. The apparatus of claim 6, wherein the actuator is one or more of a side belt and a side roller, wherein:
the side belt or the side roller are configured to contact the side of the object;
the side belt moves laterally when actuated; and
the side wheel rotates laterally when actuated.

8. The apparatus of claim 2, further comprising a non-powered support over which the object moves while within the illuminated region.

9. The apparatus of claim 8, wherein the entrance is elevated above the exit such that gravity is the sole force urging the object along the path from the entrance to the exit.

10. The apparatus of claim 8, wherein the path is configured such the object will traverse the illuminated region when the object enters the illuminated region at or above a defined minimum velocity.

11. The apparatus of claim 8, wherein the fixed support comprises a non-powered rolling element over which the object rolls.

12. The apparatus of claim 8, further comprising a tumbling actuator disposed proximate to the path and configured to reorient the object.

13. The apparatus of claim 2, further comprising a reflector disposed within the interior and configured to redirect a portion of the ultraviolet light onto a shadowed surface of the object.

14. The apparatus of claim 1, wherein the ultraviolet light is limited to wavelengths within the band of 220-225 nm such that the apparatus does not pose a hazard to people proximate to the apparatus while in operation.

15. An apparatus for disinfecting an entire external surface of a bowling ball while it is rolling along a ball return track of a bowling alley, comprising:
an enclosure configured to be disposed proximate to the track such that the track traverses an interior of the enclosure; and
a light source coupled to the enclosure and configured to emit ultraviolet light;
wherein a total energy of the ultraviolet light received at the entire external spherical surface of the bowling ball, as the bowling ball passes through the enclosure, is sufficient to inactivate at least a threshold percentage of a target infectious agent.

16. The apparatus of claim 15, further comprising a reflector coupled to the enclosure and configured to reflect a portion of the ultraviolet light toward the track so as to illuminate a portion of the bowling ball that is shadowed from the ultraviolet light coming directly from the light source.
